(19) United States
(12) Patent Application Publication
(10) Pub. No.: US 2021/0074414 A1
(43) Pub. Date: Mar. 11, 2021

(54) Electronic Informed Consent and Discharge

(71) Applicants: Syed Kamran Hasni, London, KY (US); Mary Christina Perkins, Lily, KY (US); Anita Maria Swafford, Bryant's Store, KY (US)

(72) Inventors: Syed Kamran Hasni, London, KY (US); Mary Christina Perkins, Lily, KY (US); Anita Maria Swafford, Bryant's Store, KY (US)

(21) Appl. No.: 16/953,800

(22) Filed: Nov. 20, 2020

Related U.S. Application Data
(63) Continuation-in-part of application No. 15/530,107, filed on Dec. 5, 2016.

(51) Int. Cl. G16H 40/20; G16H 10/60; G16H 10/20; G16H 70/20; G16H 20/10; G06Q 10/10; H04L 9/32; G06F 3/0482; G06F 3/16

(52) U.S. Cl. CPC G16H 40/20; G16H 10/60; G16H 10/20; G16H 70/20; G16H 20/10; H04L 51/02; G06Q 10/1095; H04L 9/3247; G06F 3/0482; G06F 3/167; G06Q 10/107

(57) ABSTRACT

A method and device for obtaining a patient's electronic informed consent for a medical procedure. The device consists of a program accessible by smart-phone, digital pad, or computer with an internet connection. The program includes all of the pertinent information regarding the medical procedure, which can be show to the patient on an embedded video or read to the patient through an audio component of the program. The program includes the ability to accept and record the patient's signature on the device and transmit that to the medical facility. The program also includes electronic discharge information to inform the patient of post procedure medication, activities, and follow-on appointments.

### CROSS REFERENCE TO RELATED APPLICATIONS

This application claims priority to, and is a continuation in part of, U.S. application Ser. No. 15/530,107, filed on Dec. 5, 2016.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

The present invention relates to a computer program and smart phone application that allows a patient to learn information about a scheduled procedure and provide their informed consent through a smart phone application.

Before a patient undergoes a medical procedure medical personnel describe the procedure to the patient, including any side effects or risks to the procedure, and then obtain the patients consent to perform the operation. Typically this is done in person, with a doctor or nurse describing the procedure and answering the patients questions. This can be time consuming for the medical personnel, and this time could be better spent on other activities. It is also well known that many patients are worried or concerned about the impending procedure and therefore find it difficult to focus on what they are being told. It is also well known that many patients are uncomfortable or intimidated by medical personnel, particularly doctors, and are often afraid or uncomfortable in asking questions about their condition and the impending procedure. There is a need, therefore, for a system that is non-intimidating, and interactive, that allows a patient to spend as much time as they need to learn about their upcoming medical procedure, and to ask questions of an impersonal program.

### SUMMARY OF THE INVENTION

The invention is a step by step walk through electronic informed consent program for medical procedures, and an incorporated electronic discharge system. It uses a computer program that can be accessed and used on a smart phone, tablet, or laptop computer. It allows a patient to view information about a medical procedure they are about to undergo. The first few pages are for the collection of demographic information about the patient. Then the application walks the patient through the procedure step by step, and has links that allow the patient to click to learn additional information. Each page of the application describes the medical procedure and any risks involved, and allows the patient to ask questions at each step to obtain additional information. At the end of the overview of the medical procedure there is a “Frequently Asked Questions” (FAQ) page about the procedure. After the patient is satisfied that they have learned all of the pertinent information about the procedure they then can go the consent page, and sign to indicate their consent. The invention also includes an electronic discharge program that explains to the patient the procedure just performed, follow on or follow up procedures and appointments, medicines prescribed for recovery, and foods and activities recommended to the patient for recovery. The electronic discharge also includes a signature acknowledgement page where the patient acknowledges receipt of the post procedure information.

The invention also includes an internal database of medical procedures which allows the device to be used for a wide variety of procedures. The specific informed consent page that the patient will go to will be linked to this database so that the patient will see the procedure that they are scheduled to undergo. This system allows the patient to learn in detail about the medical procedure that they are about to undergo, but saves time for the medical staff because they do not have to explain the procedure in detail. This device is to be used in conjunction with discussion with the doctor or other appropriate medical professional.

The disclosed invention consists of a method for electronic informed consent and electronic discharge confirmation for a patient in a health care provider system, comprising the steps of providing a computer application accessible by a smart phone or computer; wherein said application is in communication with said health care provider's medical records system and is interfaced with said patient's electronic medical records within said system; accessing said application through said smart phone or computer; inputting said patient's name to access said patient's data within said application; selecting a scheduled medical procedure from a menu within said application; viewing information regarding said scheduled medical procedure on said application; reviewing frequently asked questions regarding said scheduled medical procedure on said application; assessing informed consent information from said application; acknowledging receipt of said information regarding said scheduled medical procedure and said informed consent; signing an informed consent form embedded in said application and saving the electronic signature; and sending said electronic signature to said health care provider. It includes the additional steps of accessing said application after said scheduled procedure; reviewing information regarding the patient's diagnosis and conditions; viewing information regarding post-procedure medication, activities, appointments, and recovery plan; acknowledging receipt of said post-procedure medication, activities, appointments, and recovery plan; signing an acknowledgement form; and sending said signed acknowledgement to said health care provider. The method can also send said post procedure appointment information to said patient by e-mail with an automated calendar reminder, wherein said calendar reminder accesses said patient's computerized calendar to provide said patient with appointment reminders. In the preferred embodiment the computer application includes an audio component wherein said audio component reads the information in each of the steps so that a hearing impaired patient or a patient with limited literacy can fully understand the information.

The method for electronic informed consent and electronic discharge may also comprise the steps of: providing a computer application accessible on a smart phone or computer, wherein a patient accesses said computer application, wherein said application includes an audio component to read aloud the information on a page or the information needed to be inputted in a blank, and wherein further said application includes the option to turn off the audio component; wherein the first page of the application is a fill in the blank form for patient data including name, date of birth, address, phone number, and email address; wherein an audio component is incorporated within the first page to audibly instruct the patient to “provide name in this blank,” “provide date of birth in this blank,” etc., corresponding to each blank, when all of the required information is inserted into the appropriate blanks the patient will be able to go on to the next page, and when all of the required information is in the blanks the audible portion will say “please click next to proceed to the next screen,” the first page will also have a blank for the hospital or medical facility that will be selected by either a pull-down menu, a click through menu, or a autofill blank that completes the name of the potential hospital based on the letters the patient begins to type in, and the audible portion will read aloud each option to allow those with limited vision or reading skills to select the appropriate hospital; once this information is successfully inputed the patient will be able to click next, and the audio portion, if activated, will instruct the patient that they may now click next to proceed to the next page; the next page will be the “procedures” page which will allow access to information on the procedure that the patient will be undergoing, the procedures may be available in a number of different formats; in one format they will be in a list on the page and the patient can scroll through and select the procedure from the list, and wherein, if the audio portion is activated the application will read off each procedure; in a second option the procedure will be in a drop down menu for selection, which also has the option of having the audio portion read out the procedures to be selected; once the patient selected the desired procedure the next page will have detailed information about that procedure, wherein, in one variation there will be a video with graphic and audio information describing the procedure in detail, wherein the procedure information will describe the preparation for the procedure, including any pre-procedure diet information, information about any risks, and information about the recovery from the procedure, and wherein the audio portion can read all of this information to the patient, and wherein further the program has the ability to allow the patient to stop and rewind or scroll back to rehear information; wherein there is a help button or a floating “chat” tab to allow the patient to contact someone for additional information; once the video is complete there will be a Frequently Asked Question (“FAQ”) page, and the patient can either scroll through, or have the audio portion read the FAQs to the patient, and wherein when the information is complete the patient can click the link to the next page; and wherein on the next page there is the informed consent form page, and wherein the first step is for the patient to either read through the informed consent information or have the audio portion read the information to the patient, and when the patient is satisfied and consents to procedure they will click yes and then be taken to the signature page; wherein when the patient clicks “save” the application saves the informed consent paperwork with the patients signature in a format such as pdf, and internally sends an e-mail of the form to the patient and the appropriate doctors office; and wherein further, when the patient is ready to be released the application includes electronic discharge information, including information regarding the just completed medical procedure, diagnosis and conditions, information regarding post-procedure medication, information regarding post-procedure goals and activities, information regarding post-procedure follow-up appointments, and wherein further the patient can acknowledge receipt of the post-procedure information.

### BRIEF DESCRIPTION OF THE DRAWINGS

FIG. 1 is a screenshot of the opening page of the phone application based program.

FIG. 2 is a screenshot of the patient information page of the program.

FIG. 3 is a screenshot of the “Procedures” page of the program.

FIG. 4 is a screenshot of the selected Procedure.

FIG. 5 is a screenshot of the Frequently Asked Questions Page.

FIG. 6 is a screenshot of the patient signature selection page.

FIG. 7 is a screenshot of the patient signature page as signed by the patient.

FIG. 8 is a screenshot of the patient signature page after the patient has selected “save.”

FIG. 9 is a screenshot of the e-mail page to send the signed Electronic Informed Consent Form.

FIG. 10 is the web-based dashboard page with the procedures pull down menu opened.

FIG. 11 is the web-based procedures page with a detailed description of the procedure.

FIG. 12 is the web-based Frequently Asked Questions menu page.

FIG. 13 is the web-based patient and witness acknowledgement page.

FIG. 14 is the Electronic Discharge (“EDC”) sign in page for authorized users or health care staff.

FIG. 15 is the EDC dashboard for health care staff.

FIG. 16 is the EDC patient demographic and medical vital statistics page.

FIG. 17 is the EDC patient diagnosis and conditions page.

FIG. 18 is the patient's email input screen for medication notification.

FIG. 19 is a screenshot of a representative medication information page.

FIG. 20 is the EDC patient follow-up appointment listing page.

FIG. 21 is the EDC patient's goals activity page.

FIG. 22 is the EDC patient medications page.

FIG. 23 is the EDC patient allergy page.

FIG. 24 is the EDC patient lab results page.

FIG. 25 is the patient's electronic immunization record.

FIG. 26 is the EDC patient and witness acknowledgement page.

FIG. 27 is the EDC end page.

### DETAILED DESCRIPTION OF THE INVENTION

Detailed embodiments of the present invention are disclosed herein. It is to be understood that the disclosed embodiments are merely exemplary of the invention, and that there may be a variety of other alternate embodiments. The figures are not necessarily to scale, and some features may be exaggerated or minimized to show details of particular components. Therefore, specified structural and functional details disclosed herein are not to be interpreted as limiting, but merely as a basis for teaching one skilled in the art to employ the varying embodiments of the present invention.

The Electronic Informed Consent Application (EICA), including the Electronic Discharge (EDC) component, is a computer program that can be web based and accessed through the Internet, or incorporated into a smart phone computer application and accessed on a smart phone or similar device. In the preferred embodiment the EICA is uploaded to the Android Development Tool. It is also possible, and within the conception of the invention, to create a version of the EICA for the Apple iPhone platform. The EICA is designed to interface with, and exchange information with, a patient's electronic medical records.

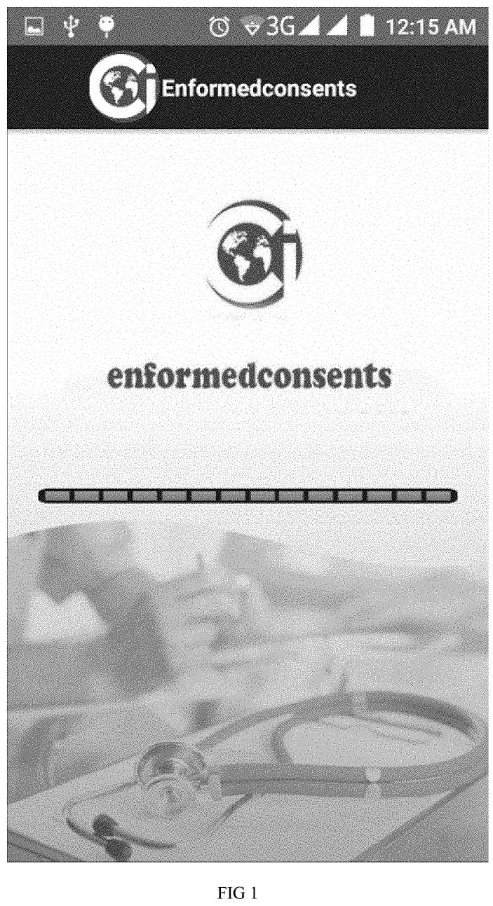

The patient uses his or her smartphone, or a smartphone or digital computer tablet or pad (such as the iPad®) provided by the hospital or other medical facility, to open the Android Studio (which houses all Android Applications) and selects the EICA, which is then loaded to the patient's phone. Once the EICA application (hereinafter “app”) is downloaded, the patient clicks the icon to open the EICA app. FIG. 1 shows the EICA smartphone opening page. In an alternate embodiment the EICA is web-based, and the patient will access the website from their phone or tablet web browser. It is also possible, and within the conception of the invention, for the patient to access the EICA on a lap top or desktop computer at the doctor's office. In that variation, once the patient has accessed the website, the screens and steps are the same as with the phone app based version.

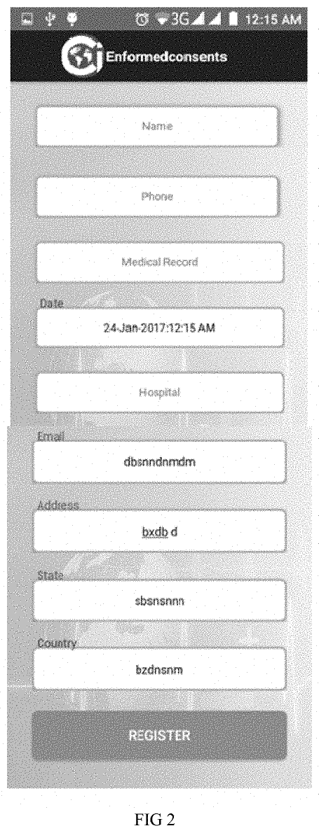

When the EICA app opens the patient will be presented with a page, shown as FIG. 2, with a fill-in-the-blank form requiring that the patient enter their name, birth date, address, phone number, and e-mail address. The page will also have either a click-though section or pull-down menu to select the hospital or other medical facility where the upcoming medical procedure will be performed. Once the patient has completed the required information they press the “send” button at the bottom of the page, which takes them to the next page.

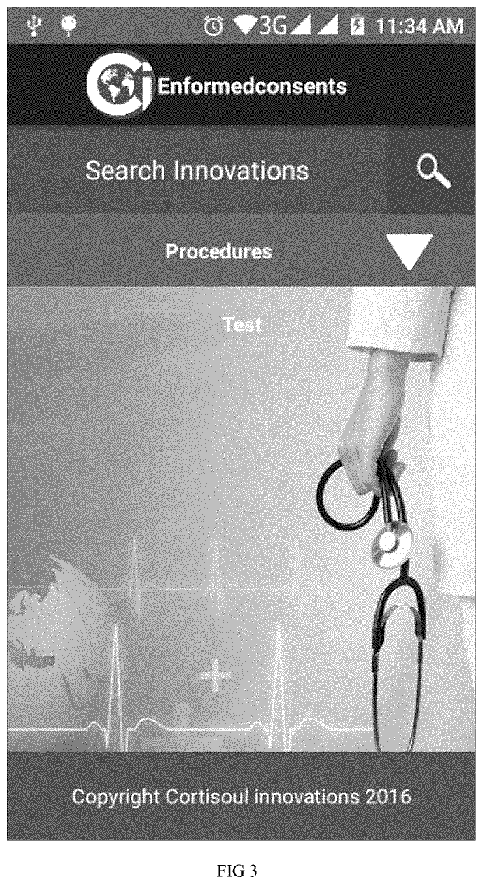

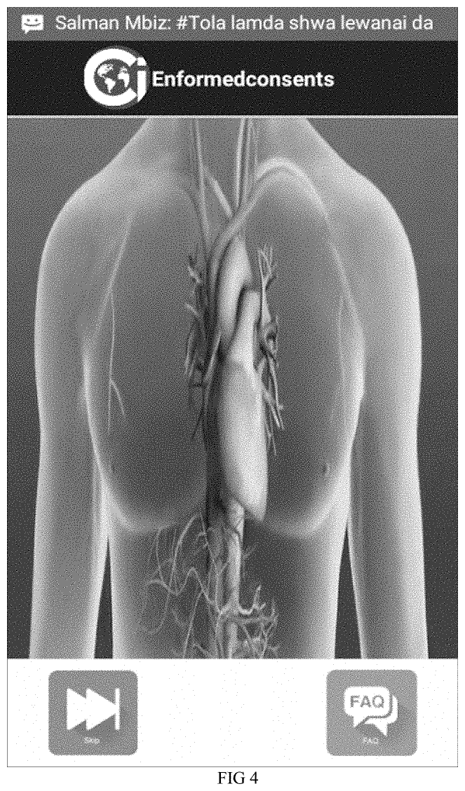

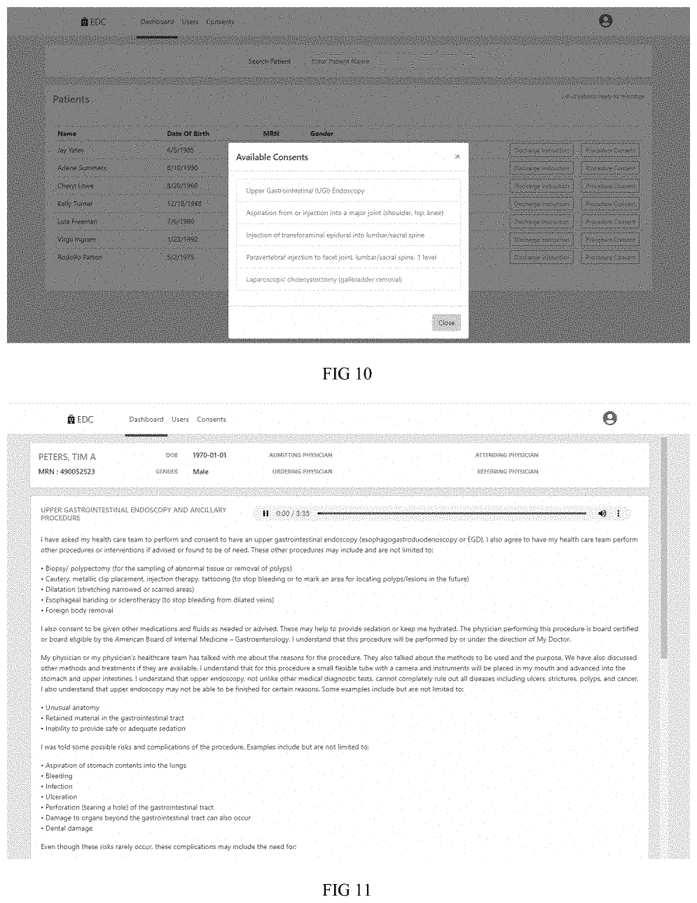

The next page is the “Procedures” page, which contains a pull-down list of the all the possible procedures available. In one embodiment these procedures and the information related to them are embedded in the EICA app, and in another embodiment the app links to a website that contains all of the information. FIG. 3 is the phone app Procedures page, and FIG. 10 is the web based version. The “Procedures” page includes a pull down menu, so when the patient clicks “Procedures” a list of medical procedures appears on the screen. The patient then scrolls through the list until they arrive at their scheduled procedure, and then they click on the procedure to learn more information about it. FIG. 4 is the phone app specific Procedure page and FIG. 11 is the web based version.

Once the user clicks their schedule procedure, information about the procedure will be shown. In one embodiment there will be a video with a fairly basic illustrated version of the procedure, showing the location on the body of the appropriate body part, a description of the malady, a description of the procedure to address the malady, information about risks related to the procedure, and recovery from the procedure. There will also be information about post-procedure information such as limits on activities post-procedure activities, care for things like stiches and the like, and information about follow up appointments. At each stage in the video the video will stop temporarily to give the patient the opportunity to rewind, or scroll back the video to see it again, or to ask questions. In one embodiment this will be asking questions of a medical personnel watching the video with the patient, and in another it will be to allow the patient to ask a question on a chat feature of the app. In an alternate embodiment the procedure will be presented in text, and the audio component will read the text aloud to the patient.

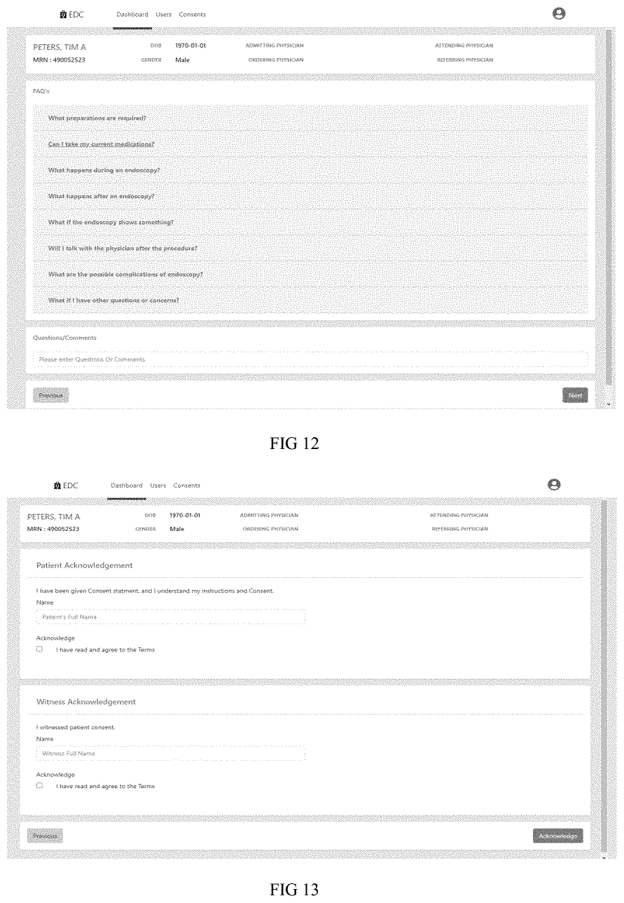

Once the video is completed there will be a “Frequently Asked Questions” (“FAQ”) page, with the answers to frequently asked questions about the medical procedure. FIG. 5 is the phone app version and FIG. 12 is the web based version. In the preferred embodiment the patient will have to go through the FAQs, and the program will read each question and each answer out loud. This is to ensure that those patients with limited literacy or visual impairments will have heard the information. Additionally, studies have shown that most people retain information better when hearing it rather than reading it. In this embodiment it will be possible for a patient to select a “silent” option and read the information themselves.

There is also a prominent “Help” button on this page that will allow the patient to find out how to use the app, the page, the video, or the Frequently Asked Questions. In one embodiment there will be a “chat” feature using Artificial Intelligence technology to answer common questions. In another embodiment there will be a live agent to answer questions. In a third embodiment the patient will be with a medical staff member such as a nurse who will be able to answer the questions live.

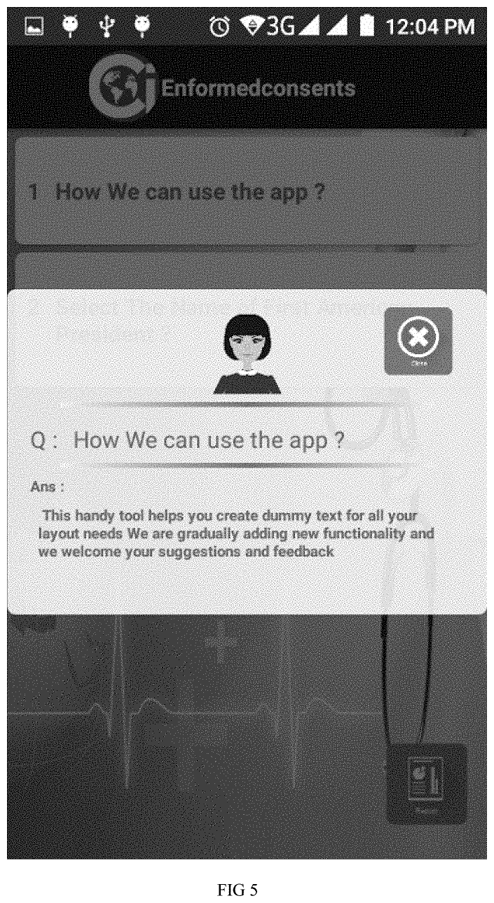

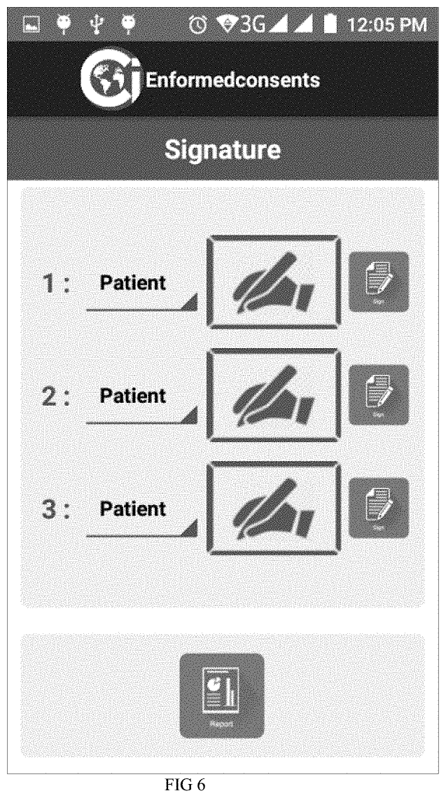

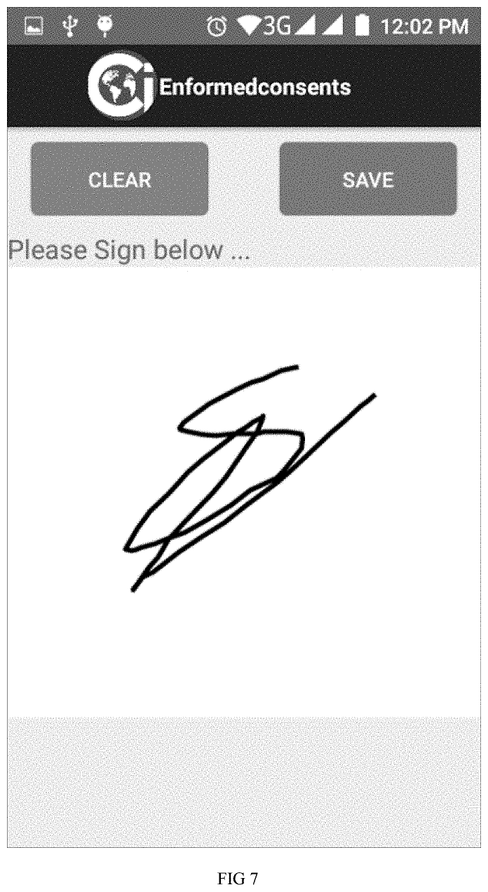

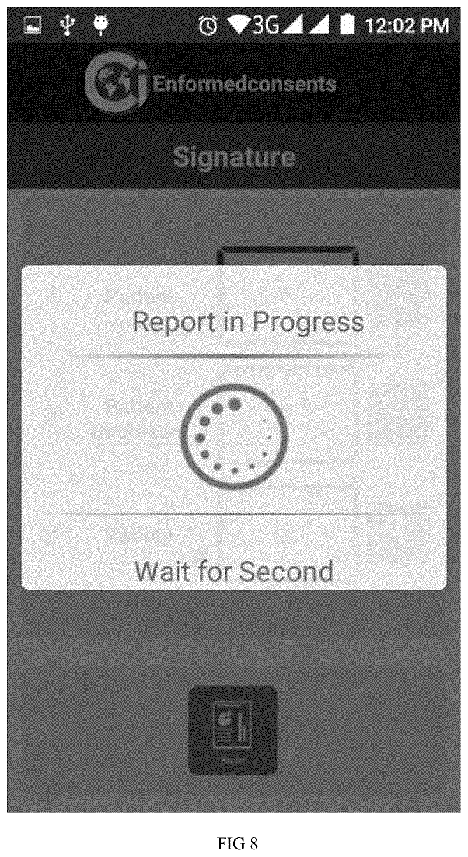

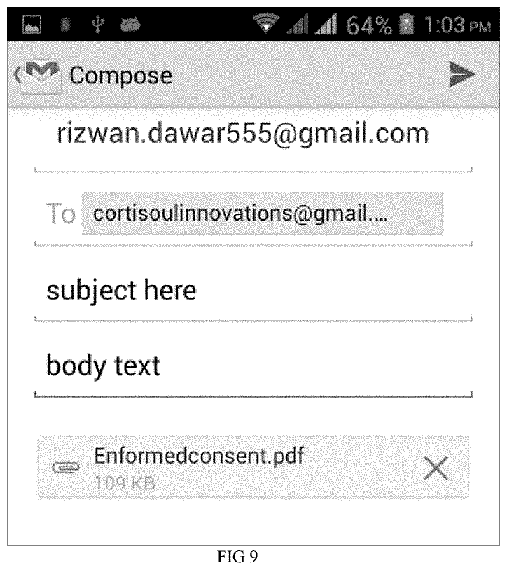

Once the patient has viewed the video, read through the frequently asked questions, and asked any questions of their own, they are then directed to the “Informed Consent” page. The first step on this page is an explanation of the Informed Consent procedure. This explains to the patient that medical personnel need a patient's consent before performing certain medical procedures, and this consent is considered “informed” when they are fully informed about their scheduled procedure. As with the FAQs, in most embodiments this feature will be presented audibly, read to the patient to ensure proper understanding. The EICA app then asks the patient if they understand the “Informed Consent” process, as shown in FIG. 6, and after the patient clicks “yes,” the app takes them to the next page which asks them if they understand their scheduled procedure. If they click “yes,” the patient is then taken to the signature page, FIG. 7 phone app, FIG. 13 web based version, where they acknowledge that they have been fully informed about their scheduled medical procedure, and they consent to allow the medical facility and personnel to perform the procedure. In one embodiment the app will allow the patient to sign with their finger or a stylus, shown in FIG. 8, and in another embodiment the patient can sign with a digital signature. Once the patient has signed they click “save,” and the documentation is saved on the app, and then transferred to the website and saved as a pdf (or similar file format) with a copy sent to the medical facility and another to the email address provided by the patient, as shown in FIG. 9.

The next step in the procedure is the Electronic Discharge (“EDC”). Upon discharge of the patient, the Electronic Informed Consent Application (EICA) shall provide the patient information about their discharge, with the second component of the invention, the electronic discharge EDC. As with the EICA, the EDC interface can be on a smart phone or tablet. In the preferred embodiment the data described below is on a web based program so that the information can be pulled up from the hospital and directly from the patients electronic medical records.

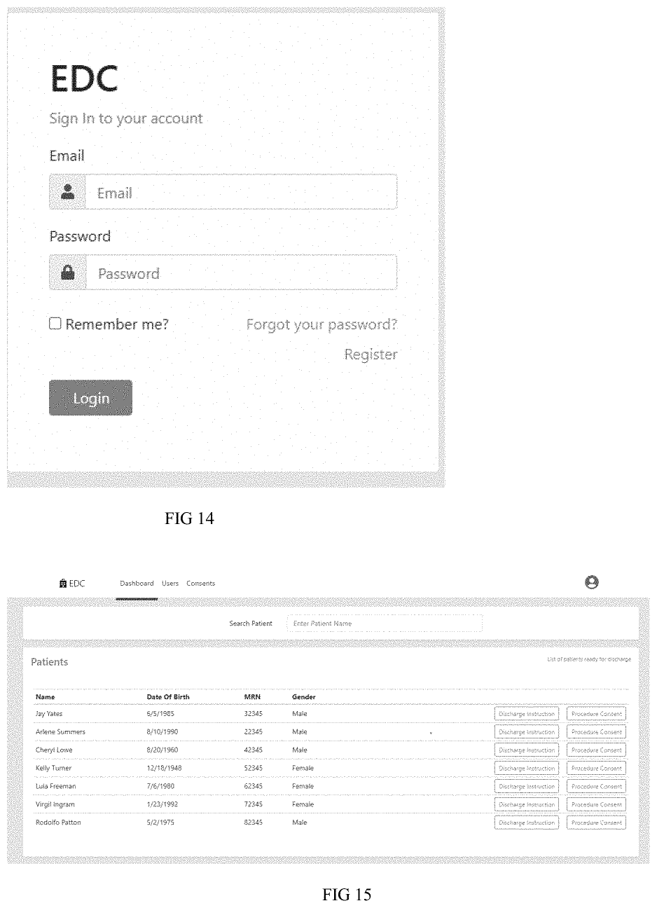

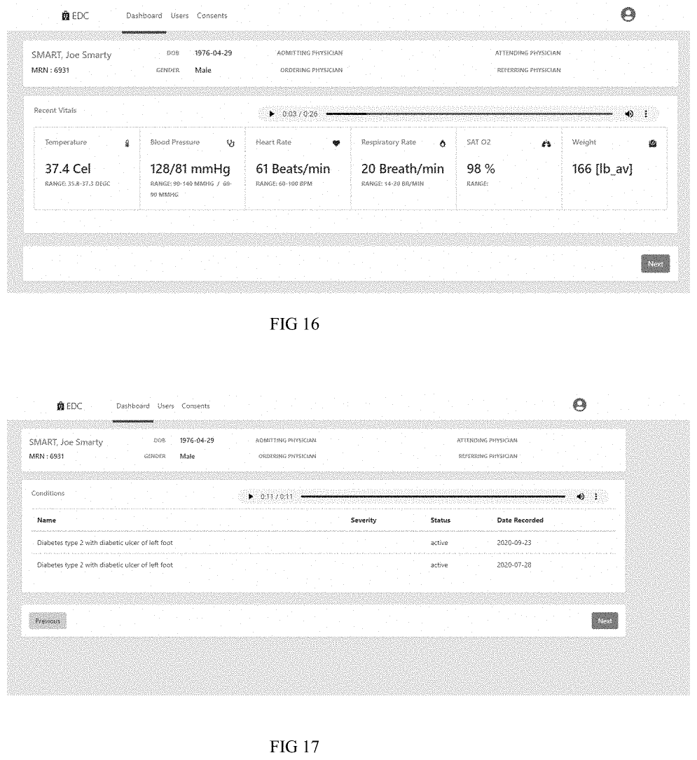

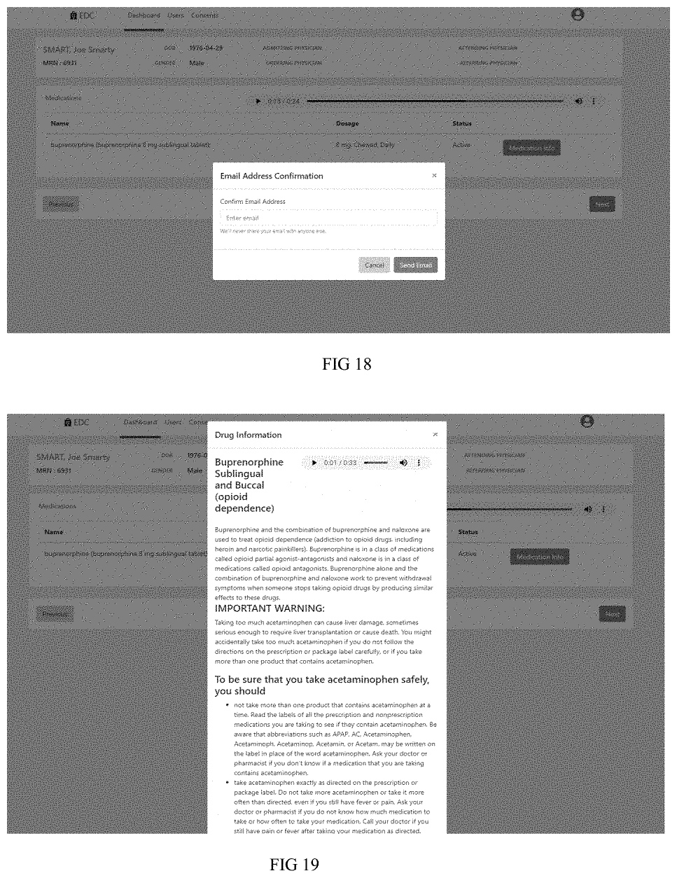

The patient opens the app by clicking the EDC icon, and once the sign-up page, as shown in FIG. 14, is open the patient begins typing either their last name or their patient ID number. It is possible, and within the conception of the invention for the EICA to use the autofill feature for patient names and patient numbers in the system. In a second embodiment, shown in FIG. 15, the hospital staff will pull up a list of patients and access the patient's information for them. Upon retrieval of the patient's data, the EDC will commence. On the first screen, FIG. 16, the patient's demographic information and most recent medical vital statistics, including blood pressure, heart rate, weight, and the like will be displayed. As with the Informed Consent information at the beginning of the patient's visit, the information on the Electronic Discharge will be audibly read to the patient, and as with the Informed Consent feature the patient will have the option of turning off this feature, though that is not recommended. When the patient has reviewed his or her vital statistics he or she can press next.

On the next screen, FIG. 17, will be information called “Diagnosis and Conditions,” which outlines the medical procedure that the patient has just undergone, the diagnosis, along with general information about the recovery prognosis. This information will have been pre-loaded from the patient's electronic medical records so that it matches the medical procedure that the patient has just undergone. After the patient is satisfied with their information they will click next to proceed to the next page.

On the next screen there will be a list of medication proscribed to the patient after the procedure, shown in FIG. 17. Each medication will be listed and described, including the required dosage and schedule for taking the medicine. There will be an email input screen so the patient can received notifications from the program related to each medication. There will also be warnings about possible major side effects to be aware of, and procedures to follow if the patient has a negative reaction to any medication, as seen in FIG. 19. The page will include information about the patient's pharmacy. After the patient has reviewed this information they will click enter and be taken to the next screen.

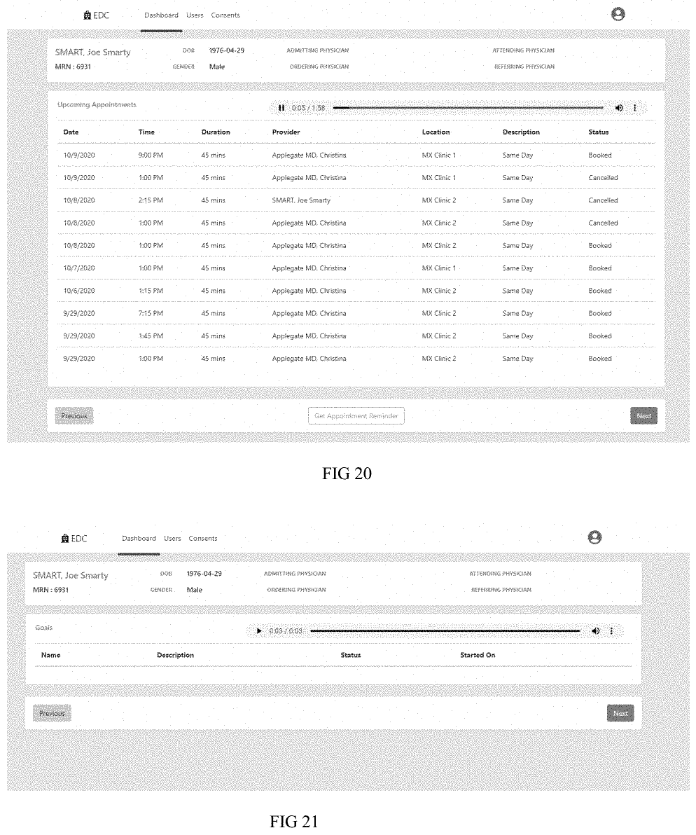

The next screen. FIG. 20, will contain upcoming appointment information, which will be read to the patient. It will describe the specific follow-on appointment and what will be done at the appointment. The patient will then enter their e-mail address and a calendar link will be sent to the patient's email address with a reminder for each appointment. If the patient is using Outlook® the program will also include an auto reminder e-mail sent to the patient's email address a day before the scheduled appointment to remind the patient of that appointment. In an alternate embodiment the EDC program will include a calendar component that can notify the patient of upcoming appointments. The EDC is also interfaced with the patient's electronic medical records as the patient's health care provider so the upcoming appointments will be in the provider's system, and typically providers send out appointment reminders. Once that step has been completed the patient will click next to go to the next screen.

The next screen, FIG. 21, will have “health goals” which will outline the patient's recovery program. One important component of these health goals is the education of the patient regarding the medical condition they are being treated for, but also education about improving overall health, not just to deal with the current malady, but to improve overall health to prevent future health related problems. The health goals will include both specific recovery goals for the patient, but also general lifestyle changes and suggestions for the patient to improve their overall health. The health goals will also include the allowed “activities” that the patient can engage in. For example, if the patient has had surgery their ability to lift heavy objects of move their body in certain ways, may be limited. The Activities page will tell the patient what they cannot do, and it will also tell them what they can do. If they are to engage in light walking or other exercise, this will be explained. This page will also contain information on diet, explaining to the patient what food and drinks they cannot consume, and what food and drinks they should consume. After the patient has read this information, or listened to it read to them, they can click next to go to the next page.

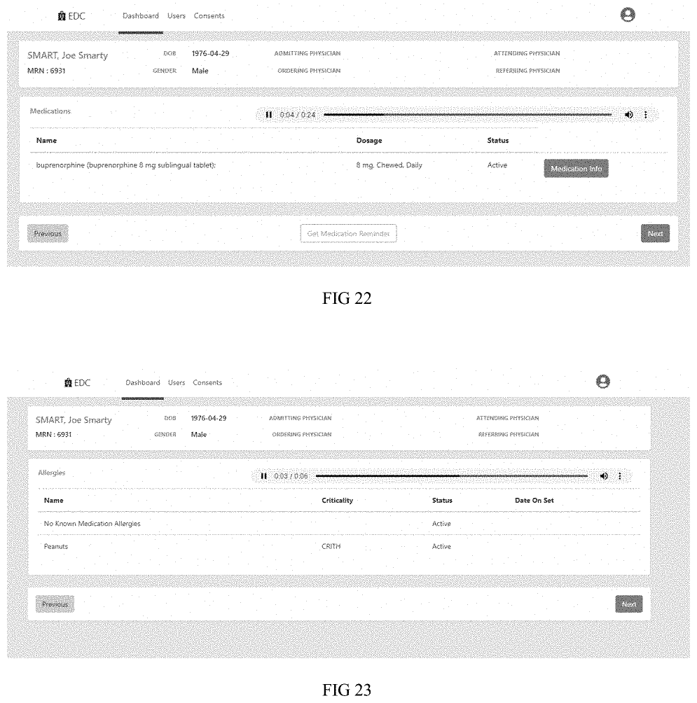

The next page, FIG. 22, will contain information about specific medications to be taken during recovery. The following page, FIG. 23 will contain Allergy information, if any. This will list the known patient allergies and explain what extra steps may be required in dealing with the allergies related to post procedure medicine or food. When the patient is satisfied and understands the information they click next.

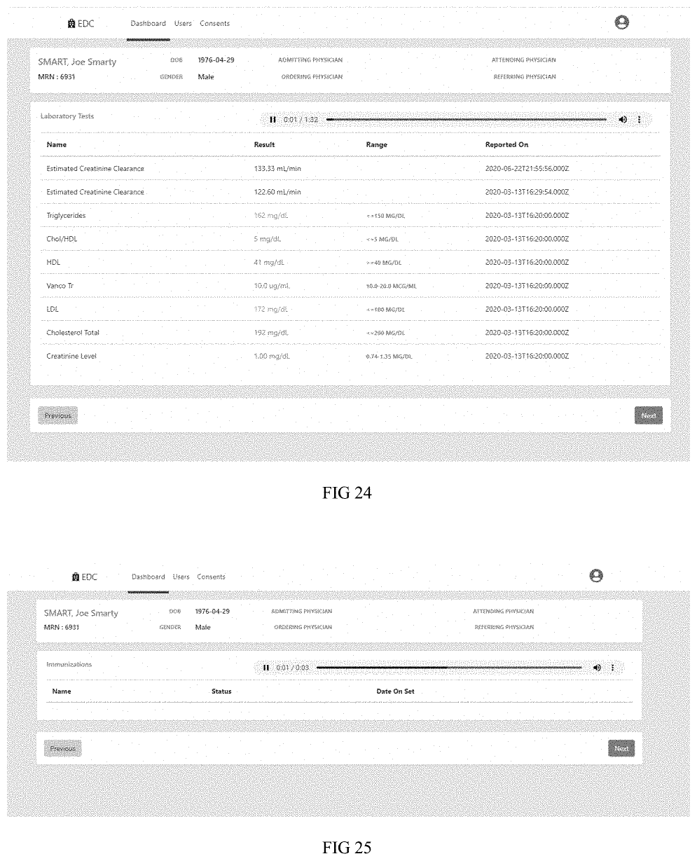

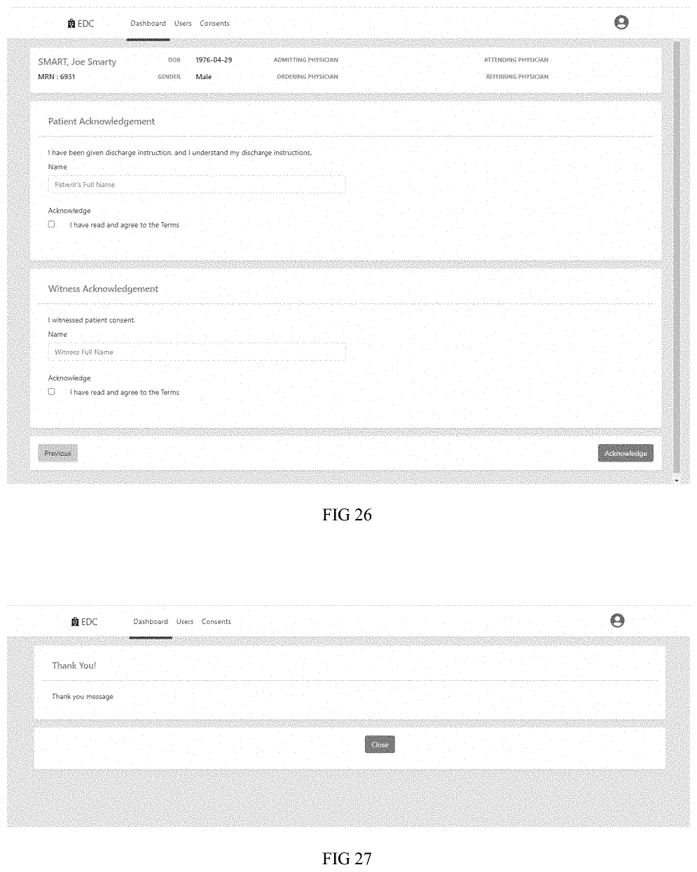

On the next page, FIG. 24, the patient's lab results will be listed. This will include information about blood work, urinalysis, cultures, biopsies and other procedure specific results. When the patient understand this information they can click next. The next screen, FIG. 25, contains information on the patient's immunization report, what they have received, what they need, and what may potentially conflict with prescribed medication. The last step is the patient acknowledgement that they have read or heard the information, understand it, and agree to it. Just as with the informed consent portion, the patient can sign directly onto the pad or screen with a finger or styles, and press accept, as shown in FIG. 26. Again, as with the EICA portion of the procedure, the acknowledgement information will be sent to the patient, along with a copy to the hospital, clinic, or doctor's office. The EDC program will include a closing page, FIG. 27, to indicate to the patient, caregiver, and medical staff, that the process has been completed.

The present invention is well adapted to carry out the objectives and attain both the ends and the advantages mentioned, as well as other benefits inherent therein. While the present invention has been depicted, described, and is defined by reference to particular embodiments of the invention, such reference does not imply a limitation to the invention, and no such limitation is to be inferred. The depicted and described embodiments of the invention are exemplary only, and are not exhaustive of the scope of the invention. Consequently, the present invention is intended to be limited only be the spirit and scope of the claims, giving full cognizance to equivalents in all respects.

We claim:

1. A method for electronic informed consent and electronic discharge confirmation for a patient in a health care provider system, said method comprising:
providing a computer application accessible by a smart phone or computer; wherein said application is in communication with said health care provider's medical records system and is interfaced with said patient's electronic medical records within said system;
accessing said application through said smart phone or computer;
inputting said patient's name to access said patient's data within said application;
selecting a scheduled medical procedure from a menu within said application;
viewing information regarding said scheduled medical procedure on said application;
reviewing frequently asked questions regarding said scheduled medical procedure on said application;
assessing informed consent information from said application;
acknowledging receipt of said information regarding said scheduled medical procedure and said informed consent;
signing an informed consent form embedded in said application and saving the electronic signature; and
sending said electronic signature to said health care provider.

2. The method for electronic informed consent and electronic discharge confirmation of claim 1 including the further steps of:
accessing said application after said scheduled procedure;
reviewing information regarding the patient's diagnosis and conditions;
viewing information regarding post-procedure medication, activities, appointments, and recovery plan;
acknowledging receipt of said post-procedure medication, activities, appointments, and recovery plan;
signing an acknowledgement form; and
sending said signed acknowledgement to said health care provider.

3. The method for electronic informed consent and electronic discharge confirmation of claim 2 including the further step of;
sending said post procedure appointment information to said patient by e-mail with an automated calendar reminder, wherein said calendar reminder accesses said patient's computerized calendar to provide said patient with appointment reminders.

4. The method for electronic informed consent and electronic discharge confirmation of claim 2, wherein said computer application includes an audio component wherein said audio component reads the information in each of the steps so that a hearing impaired patient or a patient with limited literacy can fully understand the information.

5. The method for electronic informed consent and electronic discharge confirmation of claim 2 including the further step of:
ascertaining said health care provider through a pull-down menu.

6. The method for electronic informed consent and electronic discharge confirmation of claim 2 wherein said medical procedure is selected from a pull-down menu.

7. The method for electronic informed consent and electronic discharge confirmation of claim 1 wherein said information regarding said scheduled medical procedure includes information on procedure risks and recovery rates.

8. The method for electronic informed consent and electronic discharge confirmation of claim 2 wherein, when viewing said procedure information the patient can stop and rewind to review procedure information.

9. The method for electronic informed consent and electronic discharge confirmation of claim 2 including the further steps of:
sending send post-procedure medication information to said patient by email.

10. A method for electronic informed consent and electronic discharge comprising the steps of:
providing a computer application accessible on a smart phone or computer, wherein a patient accesses said computer application, wherein said application includes an audio component to read aloud the information on a page or the information needed to be inputted in a blank, and wherein further said application includes the option to turn off the audio component;
wherein the first page of the application is a fill in the blank form for patient data including name, date of birth, address, phone number, and email address; wherein an audio component is incorporated within the first page to audibly instruct the patient to “provide name in this blank,” “provide date of birth in this blank,” etc., corresponding to each blank, when all of the required information is inserted into the appropriate blanks the patient will be able to go on to the next page, and when all of the required information is in the blanks the audible portion will say “please click next to proceed to the next screen,”
the first page will also have a blank for the hospital or medical facility that will be selected by either a pull-down menu, a click through menu, or a autofill blank that completes the name of the potential hospital based on the letters the patient begins to type in, and the audible portion will read aloud each option to allow those with limited vision or reading skills to select the appropriate hospital; once this information is successfully inputed the patient will be able to click next, and the audio portion, if activated, will instruct the patient that they may now click next to proceed to the next page;
the next page will be the “procedures” page which will allow access to information on the procedure that the patient will be undergoing, the procedures may be available in a number of different formats; in one format they will be in a list on the page and the patient can scroll through and select the procedure from the list, and wherein, if the audio portion is activated the application will read off each procedure; in a second option the procedure will be in a drop down menu for selection, which also has the option of having the audio portion read out the procedures to be selected;
once the patient selected the desired procedure the next page will have detailed information about that procedure, wherein, in one variation there will be a video with graphic and audio information describing the procedure in detail, wherein the procedure information will describe the preparation for the procedure, including any pre-procedure diet information, information about any risks, and information about the recovery from the procedure, and wherein the audio portion can read all of this information to the patient, and wherein further the program has the ability to allow the patient to stop and rewind or scroll back to rehear information;
wherein there is a help button or a floating “chat” tab to allow the patient to contact someone for additional information;
once the video is complete there will be a Frequently Asked Question (“FAQ”) page, and the patient can either scroll through, or have the audio portion read the FAQs to the patient, and wherein when the information is complete the patient can click the link to the next page;
and wherein on the next page there is the informed consent form page, and wherein the first step is for the patient to either read through the informed consent information or have the audio portion read the information to the patient, and when the patient is satisfied and consents to procedure they will click yes and then be taken to the signature page;
wherein when the patient clicks “save” the application saves the informed consent paperwork with the patients signature in a format such as pdf, and internally sends an e-mail of the form to the patient and the appropriate doctors office;
and wherein further, when the patient is ready to be released the application includes electronic discharge information, including information regarding the just completed medical procedure, diagnosis and conditions, information regarding post-procedure medication, information regarding post-procedure goals and activities, information regarding post-procedure follow-up appointments, and wherein further the patient can acknowledge receipt of the post-procedure information.

11. The method for electronic informed consent and electronic discharge of claim 10, wherein the signature page allows the patient so sign in a blank with their finger.

12. The method for electronic informed consent and electronic discharge of claim 10, wherein the signature page allows the patient so sign in a blank with a stylus.

13. The method of electronic informed consent and electronic discharge of claim 10, wherein the floating “chat” tab connects said patient to a live agent.

14. The method of electronic informed consent and electronic discharge of claim 10, wherein the floating “chat” tab connects said patient to an automated agent using artificial intelligence.
